(19) United States
(12) Patent Application Publication
(10) Pub. No.: US 2016/0317062 A1
(43) Pub. Date: Nov. 3, 2016

(54) WEARABLE MICROWAVE RADIOMETER

(71) Applicant: MMTC, Inc., Princeton, NJ (US)

(72) Inventors: Fred Sterzer, Princeton, NJ (US); Daniel D. Mawhinney, Livingston, NJ (US)

(21) Appl. No.: 15/138,698

(22) Filed: Apr. 26, 2016

Related U.S. Application Data
(63) Continuation of application No. PCT/US16/27053, filed on Apr. 12, 2016.
Application No. PCT/US16/27053 is a continuation-in-part of application No. 14/793,905, filed on Jul. 8, 2015.
Application No. 14/793,905 is a continuation-in-part of application No. 14/793,905, filed on Jul. 8, 2015.
(60) Provisional application No. 62/155,898, filed on May 1, 2015.
(60) Provisional application No. 62/155,898, filed on May 1, 2015.

(51) Int. Cl. A61B 5/05; A61B 5/00; A61N 5/02; A61B 5/01

(52) U.S. Cl. CPC A61B 5/0507; A61B 5/01; A61B 5/6804; A61B 5/4312; A61B 5/7285; A61N 5/02; A61B 5/0044; A61B 5/0042; A61B 2562/0228; A61B 2562/18

(57) ABSTRACT

Provided among other things is a wearable microwave radiometer. For example, a wearable microwave radiometer apparatus for measuring relative temperature differences comprising: (a) a circumambient garment configured to fit snugly; (b) control flat, flexible radiometer antenna(s) fitted to, or configured to fit to, the garment; (c) active flat, flexible radiometer antenna(s) fitted to, or configured to fit to, the garment, which active antenna(s) are configured in the apparatus to be positioned in a spaced-apart manner relative to the control antenna(s); and (d) a radiometer configured to monitor microwave signal from the control and active antennas and fitted to, or configured to fit to, the garment, wherein the antennas are operatively connected to, or configured to connect to, the radiometer.

This application is a continuation of PCT/US16/27053, filed 12 Apr. 2016 (the contents of which are incorporated herein in their entirety), which is a continuation-in-part of U.S. Ser. No. 14/793,905, filed 8 Jul. 2015 (the contents of which are incorporated herein in their entirety), and claims the priority of U.S. Ser. No. 62/155,898, filed 1 May 2015 (the contents of which are incorporated herein in their entirety); this application is also a continuation-in-part of U.S. Ser. No. 14/793,905, filed 8 Jul. 2015, which claims the priority of U.S. Ser. No. 62/155,898, filed 1 May 2015.

This invention was made with government support under Grant No. 1R43DK103374-01 awarded by National Institute of Diabetes and Digestive and Kidney Diseases. The government has certain rights in the invention.

The present application relates generally to wearable microwave radiometers, and uses thereof.

Any object at any temperature above absolute zero is known to emit electromagnetic radiation. This radiation is usually referred to as thermal radiation. The characteristics of this radiation depend on the temperature and properties of the object. The intensity of this thermally generated radiation is given by Planck's famous black body radiation formula that for microwave frequencies simplifies to the Raleigh-Jeans's formula, which states that the intensity of the microwave radiation is proportional to the absolute temperature and the emissivity of the object. If the variations of emissivity with temperature are small, as in the case of tissues it follows that that the microwave power emitted from tissues is directly proportional to their absolute temperature. [See article by one of the present inventors, Fred Sterzer, “Microwave Radiometers for Non-Invasive Measurements of Subsurface Tissue Temperatures”, Automedica, 1987, Vol. 8, pages 203-211]. Medical microwave radiometers are instruments that can measure the thermally generated microwave noise emissions from subsurface tissues, and then relate these measurements to the temperatures of these tissues. Such radiometers can non-invasively measure subsurface tissues temperatures to a depth of several centimeters.

What has been missing are practical apparatuses by which such microwave measurements can be tracked as a subject goes about its life. The wearable radiometer apparatus (“WRM”) described here can monitor internal body temperatures indicative of cancer activity, infection, organ inflammation, poor tissue perfusion, activated brown adipose tissues, and the like. The subject's metabolic activity can also be tracked by monitoring the temperature of brown adipose tissue.

### SUMMARY

Embodiments according to the invention include wearable radiometer apparatuses and methods of use, substantially as shown in and/or described in connection with at least one of the figures, as set forth more completely in the claims and numbered embodiments, are disclosed. Various advantages, aspects, and novel features of the present disclosure will be more fully understood from the following description and drawings.

Embodiments include, for example, a wearable microwave radiometer apparatus for measuring relative temperature differences comprising: (a) a circumambient garment configured to fit snugly; (b) control flat, flexible radiometer antenna(s) fitted to, or configured to fit to, the garment; (c) active flat, flexible radiometer antenna(s) fitted to, or configured to fit to, the garment, which active antenna(s) are configured in the apparatus to be positioned in a spaced-apart manner relative to the control antenna(s); and (d) a radiometer configured to monitor microwave signal from the control and active antennas and fitted to, or configured to fit to, the garment, wherein the antennas are operatively connected to, or configured to connect to, the radiometer.

### DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only illustrative embodiments of this invention and are therefore not to be considered limiting of its scope, for the invention may admit to other equally effective embodiments.

FIG. 1 depicts an illustrative brassiere WRM;

FIG. 2 shows an illustrative circuitry for the WRM

FIG. 3 shows microprocessor elements that can be used with the WRM;

FIG. 4 shows an illustrative WRM with multiple antennas;

FIG. 5 shows a body suit WRM;

FIG. 6 shows WRMs on two body appendages;

FIG. 7 shows a brassiere WRM with independent temperature monitors; and

FIG. 8 shows phased array for use as the active antennas.

To facilitate understanding, identical reference numerals have been used, where possible, to designate comparable elements that are common to the figures. The figures are not drawn to scale and may be simplified for clarity. It is contemplated that elements and features of one embodiment may be beneficially incorporated in other embodiments without further recitation.

### DETAILED DESCRIPTION

The WRM generally will function best when determining a relative increase or decrease in body temperature. As such, generally more than one radiometer antenna will be used, with at least one used to determine a reference microwave output from the body, which the WRM (or functionally linked computing devices) will be compared with the non-control output of the WRM.

An important advantage of wearable microwave radiometer apparatuses over conventional laboratory sized microwave radiometers is that wearable radiometer apparatuses can detect much smaller tissue temperature differences. This is because the minimum detectable temperature of a microwave radiometer is inversely proportional to the square root of the integration time and wearable radiometers can integrate over orders of magnitude longer times than conventional laboratory radiometers. Thus, when the WRM monitors an experimental location and a control location, it can for example collect data over for example 2 hours, recording for example every 60 seconds, and reliably detect temperature differences as low as about 0.1° C.

A circumambient garment is one configured to surround a portion of an animal torso, limb, head or neck. The garment can be so configured for example by being stretchable to an appropriate size, by having ties, by having loop and hook (e.g. Velcro) straps, by having straps and strap-receiving cinching fittings, and the like. Typically, one or more of the base material, straps, and the like can be stretchable such that the circumambient garment snugly biases the antennas against the animal.

In embodiments, the circumambient garment is microwave shielding garment, configured to reduce the incidence of exterior microwaves on the antenna, and optionally on the signal amplification circuits of the WRM. Microwave shielding fabrics are available for example for example as Naptex RF Fabric, from UniTech Services Group, Springfield, Mass. (available through High Ground/NSP America Inc. Charlotte. N.C.). In embodiments, microwave shielding is supplied by or supplemented by a further exterior shielding garment.

To better assure the comfort of the subject, the radiometer apparatus, exclusive of the antennas and connections to the antennas, can be for example, in embodiments, about 300 g weight or less, such as 250 g or less. Similarly to better assure the comfort of the subject, the radiometer apparatus, exclusive of the antennas and connections to the antennas, can be for example, in embodiments, about 300 cc in volume or less, such as 250 cc or less.

Exterior microwaves are those that do not come from the subject animal.

Microwaves have wavelengths ranging from one meter to one millimeter; with frequencies between 300 MHz (1 m/100 cm) and 300 GHz (1 mm/0.1 cm). For example, frequency bands of about 500 MHz can be used, such as for example from about 3.7 to about 4.2 GHz or about 1.2 to about 1.7 GHz.

The garment can have affixed to it, or can be configured to affix, one or more first locators. If the first locators are removable from the garment, the first locators and garment are configured so that the first locators can be reproducibly affixed. For example, locations for affixing can be marked on the garment, for example with markings including three or more alignment positions. The garment can have fitting that fit reciprocal fittings on the first locators.

The first locators have fittings that fit reciprocal fittings on second locators affixed to the subjects body. The fittings are such that the material of the garment cannot shift significantly from the body location dictated by the connected first and second locators. The fittings can have a snap action, or can be more easily separated by pulling the garment away from the subject body. In the latter case, a snug fit of the garment can be used to keep the connection in place.

The second locators can be initially placed by a physician, and marked on the subject's skin or hide with a permanent surgical marker. The second locators can have for example three or more notches or marks such that ink markings in the notches provide three point location. In this way, after initial placement by the physician or medical technician, the subject, or an assistant, can thereafter reproducibly place the garment for a significant period of time (the length of time until the permanent ink marking fades to much to be useful).

Surgical tape can be used to locate the garment. In this case, the garment can have markings, such as two or more, or three or more, that a physician or medical technician can mark on the subject the alignment points or lines.

In certain embodiments, the garment is a stretchable “torso” suit, meaning that it is comparable to a one-piece bathing suit. In this fashion, the antennas are more reliably kept in place without the garment riding up or down on the torso. The torso suit can be closable with a zipper, buttons, loop and hook (e.g., Velcro), or the like.

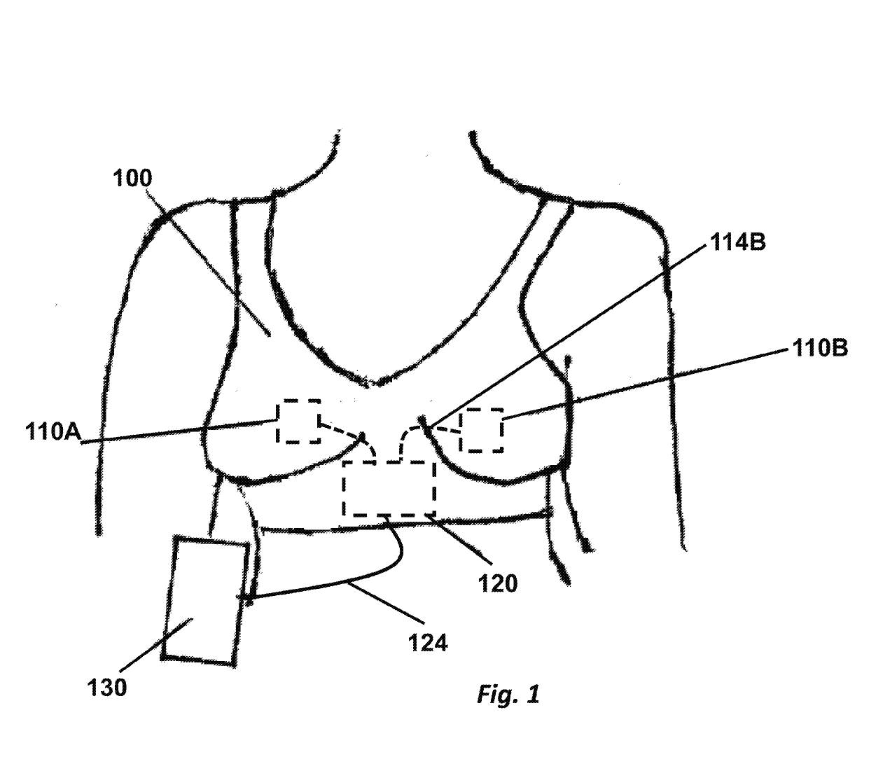

FIG. 1. shows a snug-fitting circumambient garment 100 that is a brassiere. Placed under the fabric are antennas 110A and 110B. One of these can be used as the temperature control for monitoring a medical event potentially taking place under the other. For example, the effect of an anticancer treatment on a tumor in one of the breasts can be so monitored. Radiometer 120 is used to monitor signal from the antennas 110. Radiometer 120 is generally placed under the circumambient garment. In embodiments, the garment 100 microwave shielding. In other embodiments, in use another garment (such as shirt), which garment is microwave shielding, is fitted over the garment (and the antenna). In embodiments, the second garment also fits over the radiometer. Electrical elements less in need of shielding, such as power source, DC processor, data recorder, square wave generator, or the like, can be fitted outside of shielding, such as in exterior elements case 130.

Such a brassiere can be used for example to monitor a breast tumor, with the reference antenna located at a comparable location on the non-affected breast.

The antennas for the WRM should be thin and flexible, allowing it to conform to the relevant body part. Thin, metal-clad dielectric antennas can be used.

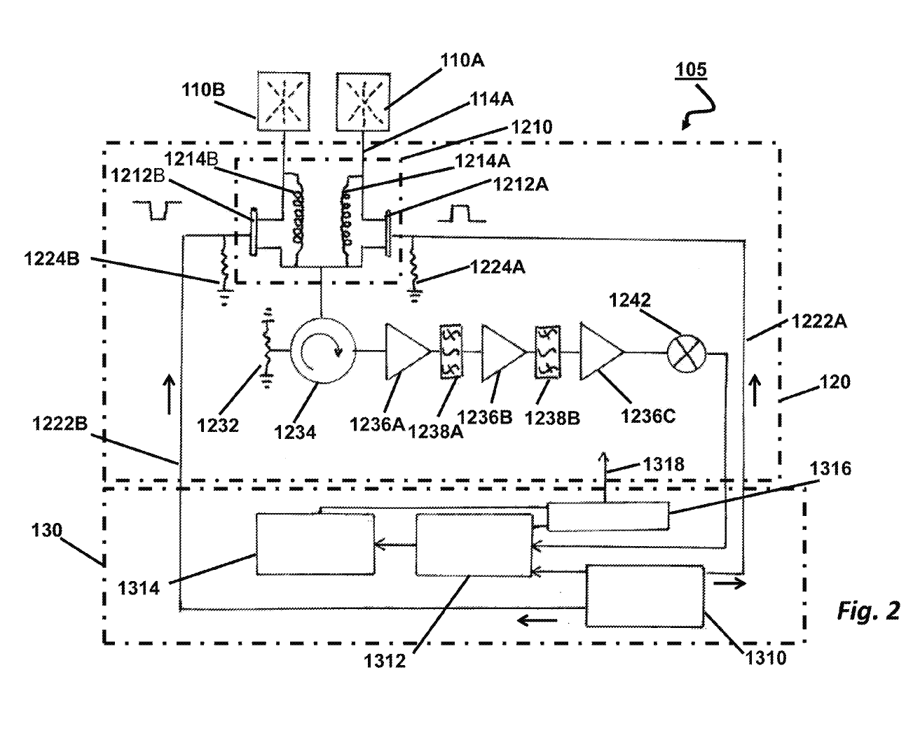

FIG. 2 illustrates an exemplary electrical schematic of a wearable medical microwave radiometer 105 that can be used as described herein. In this exemplary embodiment, wearable radiometer 105 comprises a first plurality of components that are included within an exterior elements case 130 including a square wave generator 1310 that sends complimentary square waves, alternating between a small voltage and zero, to field effect transistor (FET) gate inputs 1212A and B via electrical connectors 1322A and B (not all electrical lines are numbered for clarity). Resistors 1224A and B serve to provide a local ground return for field effect transistor (FET) gate inputs 1212A and 1212B. Not shown in the schematic in parallel with each resistor there can be a bypass capacitor to eliminate any microwave noise on the input to the FET gates. Both FET gates can be operated in a passive mode with no dc voltage applied between the drain and the source terminals. When the small voltage is applied to the FET gate, the drain to source becomes a low resistance connecting the respective antenna to the radiometer circuit. When the gate voltage is zero, the resistance is much higher, essentially disconnecting that antenna from the radiometer circuit. Each FET has an inductor (1214A and B) to parallel resonate drain to source capacitance of the FET to increase the isolation in the “off” state.

The exterior elements case can include a recorder 1314, and a power source 1316. A wearable element 120 of garment 100 can for example include a second plurality of components as will now be described. Such a recorder can be, for example, a basic recording system such as found in Holler Monitors for recording EKG's. These could be used in substantially their present form since the outputs of the WMR are similar small DC voltages. Alternatively, the WRM can include a transmitter for sending the data to an external device, such as a cell phone, tablet, or other computing device with data storage. In this latter case, the WRM can include more limited data storage in which to buffer the data until controller receives confirmation that data is externally saved.

In embodiments, the data recorder includes enough storage capacity within the WMR to store 2 days or more, or 4 days or more, or 5 days or more, of data with a sampling frequency of 30 minutes. Longer term storage is particularly useful for tracking the effects of a course of treatment, such as an anticancer treatment (or for example an infection treatment, inflammation treatment, poor tissue perfusion treatment, or the like).

The wearable element 120 of radiometer apparatus 105 according to this exemplary embodiment includes a pair of antennas 110A and 110B. Microwave energy in a given frequency band that is received by the antennas 110A and 110B is coupled to the distal end of microwave feedlines 114A and 114B, respectively. However, field effect transistors (FET) 1212A and 1212B are alternately switched between conducting and non-conducting states by the application of complementary square wave pulses supplied by square wave generator 1310. For example, when a pulse is delivered by generator 1310 to FET 1212A, FET enters a non-conducting state and emissivity correcting noise power from the terminator 1232 reflects from the open circuit and is applied to the input of the circulator 1234 to provide a relation between the measured temperature of the terminator and the output voltage it provides for calibration purposes. During that same time, no pulse is applied by generator 1310 to FET 1212B such that FET 1212B enters a conducting (“closed”) state, and this shorts the microwave feedline 1214B. As such, the microwave energy received via antenna 110B is applied to the circulator 1234. Resistors 1224A and 1224B drain the current from FETs 1212A and generator 1310.

Signal from the antennas 110A and B (e.g., slot antennas that are in direct contact with the subject) conveyed through the SPDT FET switch 1210 to microwave circulator 1234, that operates in conjunction with microwave terminator 1232, (which is heated to approximate body temperature either by heating or intimate body contact and compensates for emissivity error due to imperfect antenna/body mismatch). Microwave energy arriving at an output port of the circulator 1234 is directionally forwarded by a feedline as an input to a low noise amplifier (LNA) 1236A. The LNA 1236A amplifies microwave energy across a given frequency band and provides the amplified microwave energy to a first bandpass filter 1238A. In the illustrative embodiment a second stage of bandpass filtering is provided, such that the output of first bandpass filter 1238A is fed to the input of a second LNA 1236B the output of which is, in turn, applied to a second bandpass filter 1238B. The use of multiple filtering may be desirable where finer control over the frequency band of interest is needed for a particular application.

Signal is then directionally passed to microwave power detector 1342 (such as a wide band device, or such as a Model AD8318 microwave detector from Analog Devices, Inc., which can be tuned to a given frequency band) that produces a dc voltage essentially proportional that is directly related to the subject temperature. From microwave power detector 1342 dc signal is carried to signal processor 1312 (e.g. dc processor; or Analog to Digital Converter (ADC)). Signal processor 1312 receives signal from the square wave generator 1310 to provide the information as to SPDT switch position (e.g., which antenna is related to which dc voltage). As illustrated, the two thermal noise outputs can be measured in the same radiometer circuit by switching between the two outputs. Processed signal can be conveyed to a data storage device such as recorder 1314. Power can be provided for example by power source 1316 (e.g., battery, fuel cell). Electrical connector 1318 indicates connections to other elements.

Antennas can be connected to the patient/subject for example with removable attachment cups, similar to the way electrodes for Holter EKGs are attached, such as for example by suction or the use of adhesive. This way the antennas can be removed from the subject when the subject is washing, etc. and eventually reattached to the same marked positions.

All elements can be powered down between scans to conserve battery power. In preferred embodiments, the battery or batteries are low voltage batteries (such as, for example, from about 6 V to about 12 V), and the radiometer operates on low voltage (such as, for example, from about 6 V to about 12 V). Low voltage operation is achieved for example by using field effect transistors (FETs) for amplifiers and switches that can operate at low voltages while at the same time providing excellent microwave performance.

It will be readily appreciated that depending upon whether an n-channel or p-channel FET is used, the effects of the conductive and non-conductive states on which microwave feedline is active may be reversed. As well, other transistors and/or switching devices may be used instead.

A recording function, whether on the elements attached to the subject, or on an electronic device in communication with the elements attached to the subject, provide for storing the temperature-indicative output as a function of time.

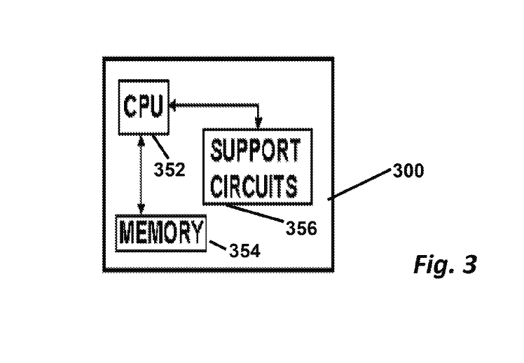

The radiometer and/or exterior elements case can incorporate electronic controllers, such as controller 300 (FIG. 3). The controller 300 comprises a central processing unit (CPU) 354, a memory 352, and support circuits 356 for the CPU 354 and is coupled to and controls one or more of the various elements of the radiometer or, alternatively, via computers (or controllers) associated with radiometer. The controller 300 may be one of any form of general-purpose computer processor that can be used for controlling various devices and sub-processors. The memory, or computer-readable medium, 352 of the CPU 354 may be one or more of readily available memory such as random access memory (RAM), read only memory (ROM), flash memory, floppy disk, hard disk, or any other form of digital storage, local or remote. The support circuits 356 are coupled to the CPU 354 for supporting the processor in a conventional manner. These circuits can include cache, power supplies, clock circuits, input/output circuitry and subsystems, and the like. Methods of operating the TNI device 100 may be stored in the memory 352 as software routine that may be executed or invoked to control the operation of the mobile radiometer, such as activating the power consuming components, and the like. Software routines may also be stored and/or executed by a second CPU (not shown) that is remotely located from the hardware being controlled by the CPU 354.

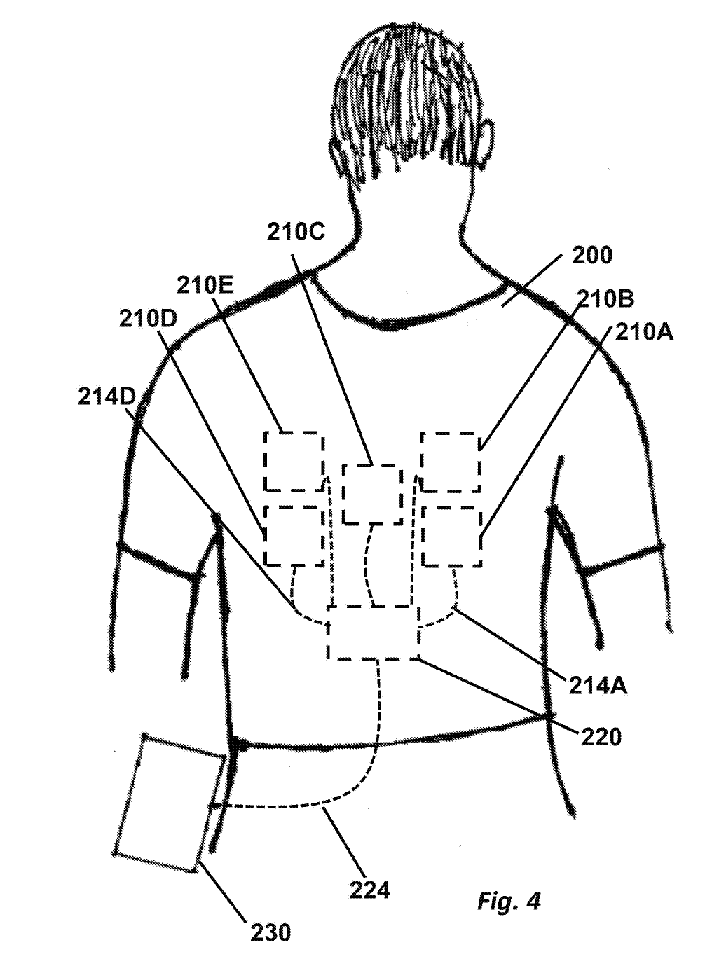

FIG. 4. shows an array of antennas 210. Multiple antennas enable the monitoring of a larger area of the subject. This can be used to produce an internal temperature map of the subject when only a specific location is of interest.

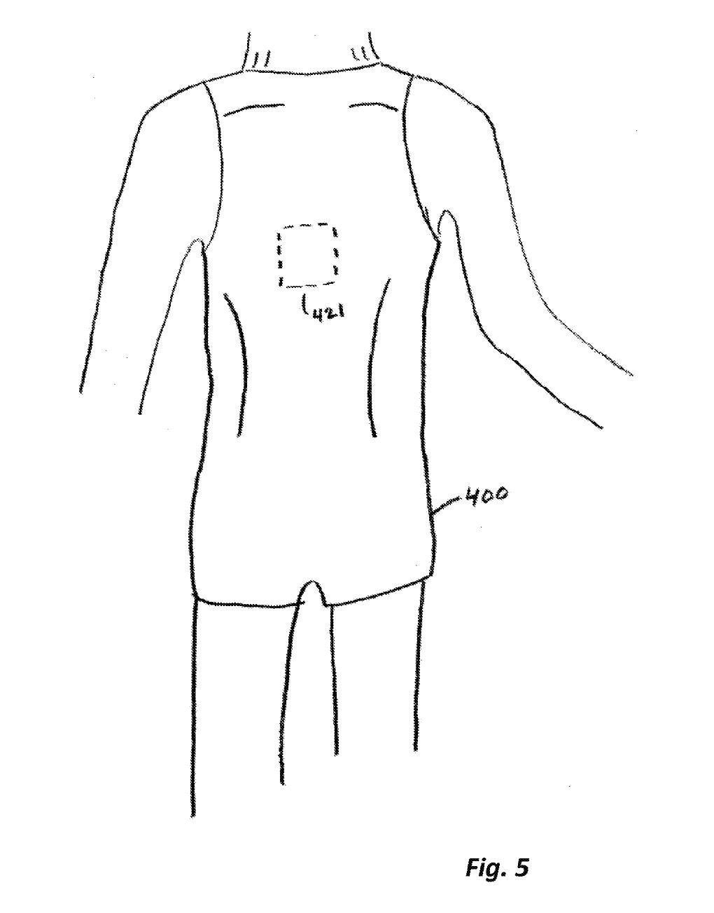

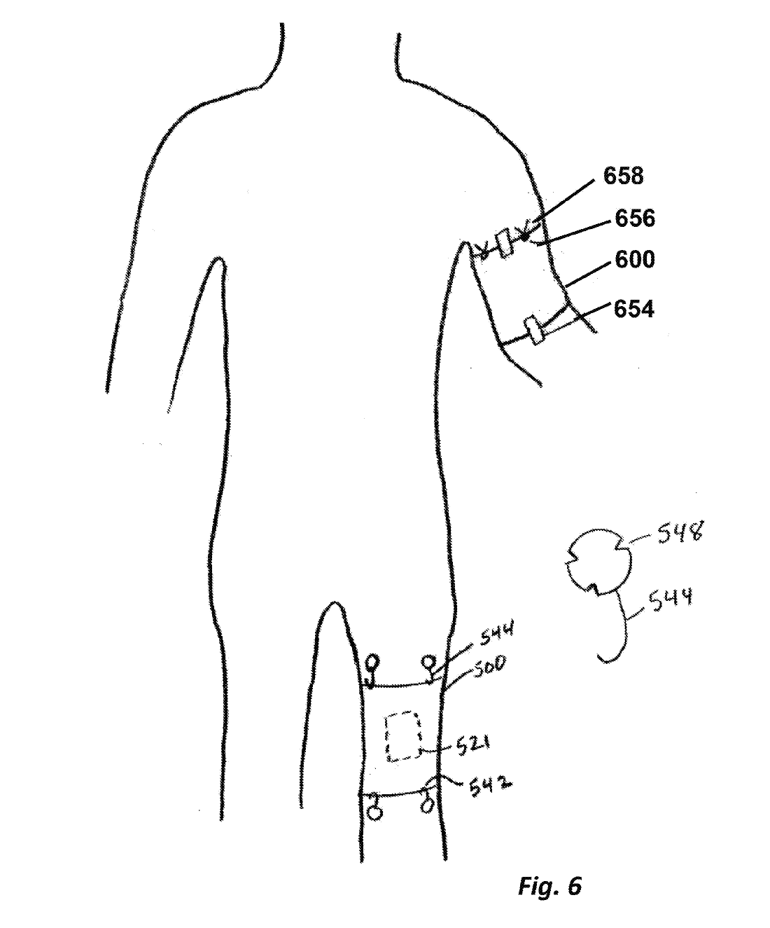

As illustrated in FIG. 5, the garment 400 can be a snug fitting torso suit. As illustrated in FIG. 6, a garment 500 or 600 can fit around an appendage. First garment locator 542 (4 occurrences) is a feature like a hole, slot, snap or the like that is configured to fit with a complementary feature of second garment locator 544. Illustrative second locator 544 has alignments marks or notches 548. These can be marked on the subject with long-lasting surgical ink, so that second locators can be reproducibly placed on a subject. Second locators can come with adhesive (for instance with a release liner fitted over the adhesive). The garment can also be secured with for example surgical tape 654. The exterior of the garment 600 can be made of a fabric that adheres the surgical tape, yet also allows for the tape to be removed without damaging the garment. Alignment mark or notch 656 can be aligned with alignment markings 658 on the subject.

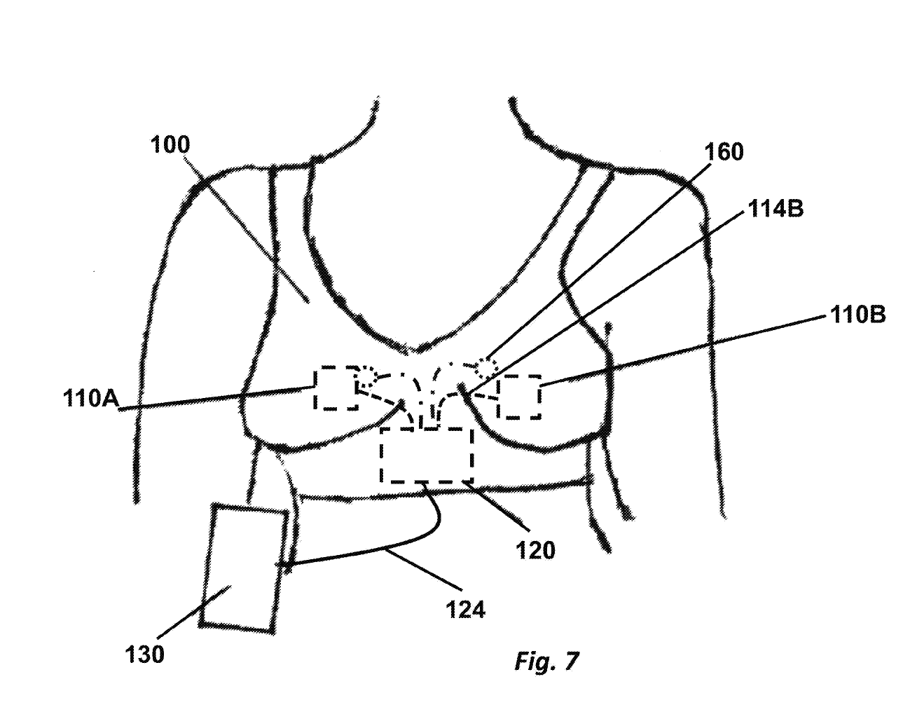

FIG. 7 is based on FIG. 1, but adds temperature monitors 160 (e.g., thermocouples or thermistors), located under the garment 100. The independent temperature monitors 160 serve to, among other things, further distinguish interior from exterior temperature and/or provide skin temperature readings to augment internal temperature measurements. When such monitors, such as thermistors or thermocouples are in direct contact and insulated from ambient conditions, meaningful data related to internal temperatures may be computed.

One of the challenges in designing a wearable microwave radiometer is power consumption. As such, The high energy consuming components of radiometer can generally only be operated episodically. Thus, in embodiments, the WRM can be programmed to operate in conjunction with input on when it will be useful to operate the scanning function of the WRM.

In embodiments, the WRM's programming can be set to alter the scanning frequency to a lower frequency when prior data indicates a slow rate of change in the monitored temperature (microwave output). The indication can be a change in the last two readings less than a prescribed cutoff, or can be based on a longer trend indicating a reduced rate of change.

In embodiments, the WRM's programming can be set to alter the scanning frequency to a higher frequency when prior data indicates an increased rate of change in the monitored temperature (microwave output). The indication can be a change in the last two readings greater than a prescribed cutoff, or can be based on a longer trend indicating an increased rate of change.

Activating brown adipose tissue (“BAT”) is believed to be a useful way to increase calorie consumption. Activated BAT consumes fat from normal white fat tissue. Current efforts to develop BAT stimulation regimes are hindered by the inability to continuously monitor BAT activity.

Exercise and bioactive molecules have been used to increase BAT activity. BAT is found for example in the upper back of a human. An antenna of the WRM can be placed over the BAT, and a reference antenna placed elsewhere. With the location controlling garments of the invention, when a good monitoring location has been found, it can be re-utilized when a subject puts the WRM back on after for example sleeping, bathing, or otherwise removing the WRM.

In embodiments, the WRM is operatively linked to another activity monitor, such as a GPS, pedometer, heart rate monitor, or the like. When the subject's physical activity increases beyond a threshold, a software protocol triggers the WRM to scan for differential temperature (via microwave output) more frequently, such as from twice an hour to 12 times an hour. In embodiments, the WRM (which can for this purpose include linked computational devices) can provide a score for BAT activation associated with a burst of physical activity. If several bursts of physical activity are close enough linked in time to be generally designated a workout, a score can be provided for the associated bursts of activity.

The WRM in embodiments has inputs for triggering when to start monitoring and/or how frequently to monitor. For example, a small electronic device such as a smart phone can be radio or otherwise linked to the rest of the WRM (in this case the WRM includes the external device) and include and app for activating scanning and/or setting the frequency of scanning. In the case for example of BAT activated with a bioactive agent, or the case of using an antimicrobial or anticancer drug, knowledge of the typical pharmacokinetics can dictate what scanning schedule will be most useful.

Where the WRM has an external input electronic device, the device can have an interface by which to input food intake, such that dietary influences on the phenomenon being monitored can be tracked.

In monitoring infection, it may be that a longer term scanning protocol (fewer scans over a longer time period) or a shorter term scanning protocol is desired. For example, for a kidney infection it may be that one wants relatively rapid feedback on whether the infection is susceptible to the antimicrobial agent used. If for example historical studies show for agent A that an effect is seen in 4 hours, then if no effect is seen in that time, the treatment can transition to another antimicrobial agent (such as with a different treatment spectrum).

Monitoring a tumor can be a longer term process. The monitoring can involve monitoring in conjunction with an anti-tumor agent or treatment. A tumor showing indications of going into remission can be expected to produce less heat by reason of less metabolic activity, and/or by reason of less blood perfusion.

In embodiments, the WRM is used to detect the heat associated with an appendicitis, for instance in a doctor's office or triage facility.

In embodiments, the WRM is used to detect and quantify low blood perfusion, low circulation, in a body part such as a foot, leg, hand, arm, heart or portion of the brain (subject for example to injury or stroke). The availability of the device to be used in ordinary life provides information on activities that improve perfusion, and can provide data on the timeline of deterioration—which can implicate a need for a change in therapy. As indicated, the perfusion measurement can be used to monitor the effectiveness of a therapy for low perfusion/circulation.

In embodiments, the WRM is used to detect cancer, such as without limitation cancer of the breast, esophagus, thyroid, lung, prostate, colon, rectum, ovaries, testes, kidney, skin, muscle, gall bladder, endometria, pancreas, and the like.

In all the disease or calorie consumption monitorings described herein, the monitoring can be used to evaluate the effectiveness of a treatment protocol, or of a lifestyle change (food, exercise, sleep, etc.)

In embodiments, the WRM scans operate at multiple frequency ranges, such that the heat from various depths can be determined, as described in as described in Fred Sterzer “Microwave Radiometers for Measurements of Subsurface Tissue Temperatures”, Automedica, 1987, Vol. 8, pages 203-211. Broadband antennas, or antennas match to different frequency ranges, can be used in these embodiments.

In embodiments, the WRM produces an visual output showing monitored temperature vs. time. The temperature can be shown for example as a height, or by a color scale. In embodiments, the temperature is shown in relation to one or more other parameters, such as heart rate, movement rate, food or drug administration, or the like.

In embodiments wherein the patient will be monitored for 24 hours without battery change, scanning frequency can be in embodiments 1 scan per 10 minutes or longer (such as 30 minutes, 60 minutes, 120 minutes).

Subjects for study with the WRM will often be humans, but can be any animal.

The WRM can also incorporate the ability to send microwaves into a subject's body, such as to heat a tumor, cancerous lesion, or other hyperplasia. The temperature elevation of such a lesion can be in the range of about 2° C. to about 5° C. above normal body temperature for the subject animal. The electrical features for providing such heating microwaves are described for example in U.S. Pat. No. 4,632,127 (issued Dec. 30, 1986), which is incorporated herein in its entirety. It is believed that elevating tumor temperatures leads to increased tumor blood flow and increased tumor oxygenation. It is believed that because of increased blood flow more systemic chemotherapeutic agents enter the tumor, and the anticancer activity of several chemotherapeutic agents increases with temperature. Increased tumor oxygenation also, it is believed, increases the effectiveness of radiation therapy.

By utilizing a phased array of antennas, the phase offsets that obtain the greatest temperature for the lesion can be used to locate the center of the lesion in x, y and depth. The search can be framed in terms of the implied x, y and depth rather than the phase offsets per se. Searches can be conducted, for example by hand, by amplifying the combined thermal noise output, rectifying it, and displaying the result on a meter. The location so determined can be used to direct the phase shifts for microwave heating energy. If utilizing the same antennas for heating and measuring, using switches to toggle between the two modes, the phase shifts from measuring can be directly used.

For this phased array, for example, several small printed circuit transmitting antennas (e.g., printed circuit X-slot micro-strip antennas) can be connected in parallel and placed around the surface location corresponding to the internal lesion that is to be heated. (These can also be used in the invention as equivalent to one large antenna but more easily placed in contact with the skin.) The frequency band of the radiometer in the transmitting mode should be centered about the heating frequency. The heating power is to be turned OFF when the radiometer is measuring tumor temperatures. A feedback circuit from the radiometer, for example via the controller, assures the measured tumor temperatures are kept close to a preset value. A phase shifter (line stretcher) after each antenna can be used to obtain the phase shift. Adjust the phase shifters behind the radiometer antennas to obtain a maximum temperature reading of the tumor. This procedure will optimize directing the heating power to the metabolically active malignant tumor whose temperature is elevated.

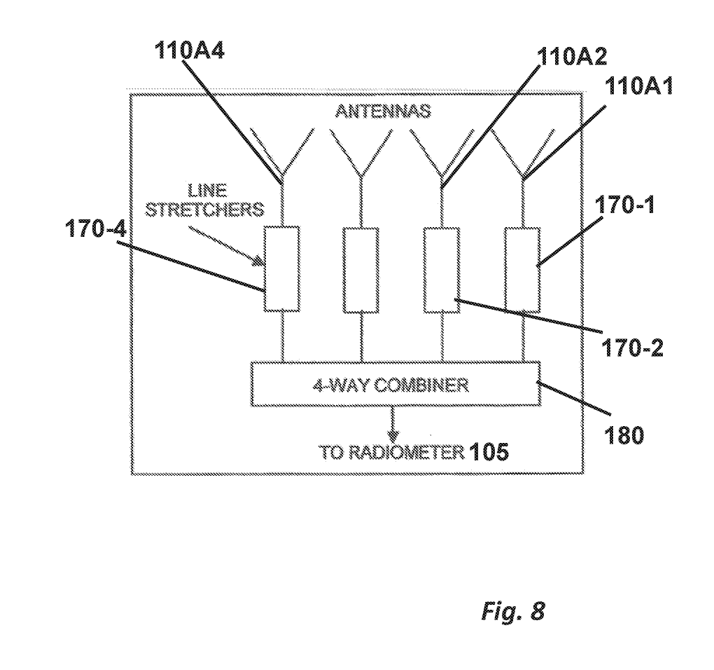

For example, as shown in FIG. 8, Antennas 110A1 to 100A4 can be used in place of antenna 110 A. Adjustable line stretchers 170-1 to 170-4 can be used to establish the phase separation. Combiner 180 is used to provide appropriate connection to the radiometer.

All ranges recited herein include ranges therebetween, and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4 or more, or 3.1 or more.

Numbered elements in the figures are as follows:

TABLE
Numbering Key

| | |
|---|---|
| 100 | Garment |
| 105 | Radiometer apparatus |
| 110A-B | Antenna |
| 114A-B | Electrical connection |
| 120 | Radiometer |
| 124 | Electrical connection |
| 130 | Optional exterior elements case |
| 160 | Thermocouple or thermistor, with electrical connection |
| 170 | Line stretcher |
| 180 | Combiner |
| 200 | Garment |
| 210A-E | Antenna |
| 214A-E | Electrical connection |
| 220 | Radiometer |
| 224 | Electrical connection |
| 230 | Optional exterior elements case |
| 300 | Controller |
| 352 | Memory |
| 354 | CPU |
| 356 | Support circuits |
| 400 | Garment (torso suit) |
| 421 | Radiometer (antennas not shown for simplicity) |
| 500 | Garment |
| 521 | Radiometer (antennas not shown for simplicity) |
| 542 | First garment locator |
| 544 | Second locator |
| 548 | Marks or notches for alignment |
| 600 | Garment (radiometer, etc., not shown) |
| 654 | Surgical tape |
| 656 | Alignment mark or notch |
| 658 | Alignment markings on patient |
| 1310 | Square wave generator |
| 1312 | Signal processor |
| 1314 | Recorder |
| 1316 | Power source |
| 1318 | Electrical connector |
| 1322A, B | Electrical connector |
| 1210 | Spot FET switch |
| 1212A, B | Switch |
| 1214A, B | Inductor |
| 1224A. B | Resister |
| 1232 | Microwave terminator |
| 1234 | Temperature circulator |
| 1236A-C | Low noise amplifier |
| 1238A, B | Band pass filter |
| 1342 | Microwave power detector |

The Invention can be further described with reference to the following numbered embodiments:

### Embodiment 1A

A wearable microwave radiometer apparatus for measuring relative temperature differences comprising: (a) a circumambient garment configured to fit snugly; (b) control flat, flexible radiometer antenna(s) fitted to, or configured to fit to, the garment; (c) active flat, flexible radiometer antenna(s) fitted to, or configured to fit to, the garment, which active antenna(s) are configured in the apparatus to be positioned in a spaced-apart manner relative to the control antenna(s); and (d) a radiometer configured to monitor microwave signal from the control and active antennas and fitted to, or configured to fit to, the garment, wherein the antennas are operatively connected to, or configured to connect to, the radiometer.

### Embodiment 1 B

A wearable microwave radiometer apparatus for measuring relative temperature differences comprising: a circumambient garment configured to fit snugly; two flat, flexible radiometer antennas fitted to, or configured to fit to, the garment in a spaced-apart manner; and a radiometer fitted to, or configured to fit to, the garment, wherein the antennas are operatively connected to, or configured to connect to, the radiometer.

### Embodiment 2

The wearable radiometer apparatus of embodiment 1A or 1B, wherein the garment comprises microwave shielding configured to isolate the antennas from exterior microwaves.

### Embodiment 3

The wearable radiometer apparatus of embodiment 1A, 1B or 2, wherein the garment has fitted to it, or configured to fit to it, one or first garment locators, the locators configured to lock into cooperative second locators.

### Embodiment 4

The wearable radiometer apparatus of embodiment 1A, 1B, 2 or 3, wherein an outer side of the garment comprises two or more locations that reversibly accept surgical tape.

### Embodiment 5

The wearable radiometer apparatus of embodiment 1A, 1 B, 2-3 or 4, wherein the garment is in the form of a brassiere with two breast cups, and wherein the antennas are configured to fit in separate cups.

### Embodiment 6

The wearable radiometer apparatus of embodiment 1A, 1 B, 2-4 or 5, wherein the garment is a stretchable torso suit, and wherein the antennas are configured to monitor separate locations on the torso.

### Embodiment 7

The wearable radiometer apparatus of embodiment 1A, 1 B, 2-5 or 6, further comprising a data recorder for recording as a function of time the microwave data from the radiometer.

### Embodiment 8

A wearable brown adipose tissue activity monitor comprising: the radiometer apparatus of embodiment 1A, 1B, 2-6 or 7; a separate physical activity monitor; wherein the radiometer is operatively connected to the activity monitor, and wherein the scanning rate of the radiometer is adjusted based on the level of physical activity determined by the physical activity monitor. Operatively connected means that data from the activity monitor is configured to effect the operation of the radiometer, for example by going to a controller or common controller.

### Embodiment 9

A kit comprising: the wearable radiometer apparatus of embodiment 3, wherein one or more one or more first locators are non-removably fitted to it; and second locators configured to adhere to a subject, the number of second locators in excess of the number of first locators.

### Embodiment 10

A kit comprising: the wearable radiometer apparatus of embodiment 8, wherein one or more one or more first locators are non-removably fitted to it; and second locators configured to adhere to a subject, the second locators having marks or notches configured to provide two point or more (or three point or more) alignment with markings on a subject.

### Embodiment 11

A kit comprising: the wearable radiometer apparatus of embodiment 1A, 1 B, 2-6 or 7; and a microwave shielding garment configured to fit thereover.

### Embodiment 12

A method of monitoring the activity of brown adipose tissue comprising: fitting the garment of the wearable radiometer apparatus of embodiment 1A, 1 B, 2-6 or 7 on a subject with one or more of the antennas of the radio fitted to detect radiation from the brown adipose tissue and one or more other such antennas fitted to detect radiation from tissue that is not brown adipose tissue; recurrently activating the radiometer to take readings of the temperature associated with the brown adipose tissue; and outputting from the wearable radiometer a representation of the temperature associated with the brown adipose tissue over time. In this and other embodiments, the representation can be a data file, an image, a spreadsheet, or the like.

### Embodiment 12A

The method of embodiment 12, further comprising: operating a separate physical activity monitor of the subject; and adjusting the timing of the activating of the radiometer based on the physical activity detected by the separate physical activity monitor.

### Embodiment 12B

The method of embodiment 12 or 12A, further comprising inputting into the wearable radiometer data concerning the timing and details of medications or dietary consumption, wherein the representation of the temperature associated over time further includes a representation of such data.

### Embodiment 13

A method of monitoring the metabolic activity of cancer tissue comprising: fitting the garment of the wearable radiometer apparatus of embodiment 1A, 1 B, 2-6 or 7 on a subject with one or more of the antennas of the radio fitted to detect radiation from the cancer tissue and one or more other such antennas fitted to detect radiation from tissue that is not the cancer tissue; recurrently activating the radiometer to take readings of the temperature associated with the cancer tissue; and outputting from the wearable radiometer a representation of the temperature associated with the cancer tissue over time.

### Embodiment 14

The method of embodiment 13, wherein the cancer is breast cancer.

### Embodiment 15

The method of embodiment 13, wherein the cancer is cancer of the esophagus, thyroid, prostate, rectum, ovaries, testes, kidney, skin or muscle.

### Embodiment 16

The method of embodiment 13, wherein the cancer is cancer of the lung, colon, gall bladder, endometria or pancreas.

### Embodiment 16A

The method of embodiment 13-15 or 16, further comprising inputting into the wearable radiometer apparatus data concerning the timing and details of medications or dietary consumption, wherein the representation of the temperature associated over time further includes a representation of such data.

### Embodiment 17

A method of heating a lesion that is a tumor, cancer or other hyperplasia comprising: fitting the garment of the wearable radiometer apparatus of embodiment 1A, 1 B, 2-6 or 7 on a subject with a phased array of active antennas of the radiometer apparatus fitted to detect radiation from the lesion and one or more control antennas fitted to detect radiation from tissue that is not the cancer tissue; determining a location for the lesion by recurrently activating the radiometer utilizing different phase offsets with the phased array to take readings of the temperature associated with the lesion and determine phase offsets that obtain a relative high value for that temperature; and directing microwave energy to the so determined location of the lesion with a phased array of antennas (which can be the active antennas).

### Embodiment 17A

The method of embodiment 17, wherein the cancer is breast cancer.

### Embodiment 17B

The method of embodiment 17, wherein the cancer is cancer of the esophagus, thyroid, prostate, rectum, ovaries, testes, kidney, skin or muscle.

### Embodiment 17C

The method of embodiment 17, wherein the cancer is cancer of the lung, colon, gall bladder, endometria or pancreas.

### Embodiment 17D

The method of embodiment 17, 17A, 17B or 17C, further comprising inputting into the wearable radiometer apparatus data concerning the timing and details of medications or dietary consumption, wherein the representation of the temperature associated over time further includes a representation of such data.

### Embodiment 18

A method of monitoring the perfusion of tissue susceptible to poor blood circulation comprising: fitting the garment of the wearable radiometer apparatus of embodiment 1A, 1 B, 2-6 or 7 on a subject with one or more of the antennas of the radio fitted to detect radiation from the susceptible tissue and one or more other such antennas fitted to detect radiation from tissue that is not the susceptible tissue; recurrently activating the radiometer to take readings of the temperature associated with the susceptible tissue; and outputting from the wearable radiometer a representation of the temperature associated with the susceptible over time.

### Embodiment 19

The method of embodiment 18, wherein the susceptible tissue is heart muscle tissue.

### Embodiment 20

The method of embodiment 18, wherein the susceptible tissue is brain tissue.

### Embodiment 21

The method of embodiment 18-19 or 20, further comprising: operating a separate physical activity monitor of the subject; and adjusting the timing of the activating of the radiometer based on the physical activity detected by the separate physical activity monitor.

### Embodiment 21A

The method of embodiment 18, 19-20 or 21, further comprising inputting into the wearable radiometer apparatus data concerning the timing and details of medications or dietary consumption, wherein the representation of the temperature associated over time further includes a representation of such data.

Where a sentence states that its subject is found in embodiments, or in certain embodiments, or in the like, it is applicable to any embodiment in which the subject matter can be logically applied.

This invention described herein is of a WRM and methods of using the same. Although some embodiments have been discussed above, other implementations and applications are also within the scope of the following claims. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

What is claimed is:

1. A wearable microwave radiometer apparatus for measuring relative temperature differences comprising:
a circumambient garment configured to fit snugly;
control flat, flexible radiometer antenna(s) fitted to, or configured to fit to, the garment;
active flat, flexible radiometer antenna(s) fitted to, or configured to fit to, the garment, which active antenna(s) are configured in the apparatus to be positioned in a spaced-apart manner relative to the control antenna(s); and
a radiometer configured to monitor microwave signal from the control and active antennas and fitted to, or configured to fit to, the garment, wherein the antennas are operatively connected to, or configured to connect to, the radiometer.

2. The wearable radiometer apparatus of claim 1, wherein the garment comprises microwave shielding and configured to isolate the antennas from exterior microwaves.

3. The wearable radiometer apparatus of claim 1, wherein the garment has fitted to it, or configured to fit to it, one or first garment locators, the locators configured to lock into cooperative second locators.

4. The wearable radiometer of claim 1, wherein an outer side of the garment comprises two or more locations that reversibly accept surgical tape.

5. The wearable radiometer apparatus of claim 1, wherein the garment is in the form of a brassiere with two breast cups, and wherein the antennas are configured to fit in separate cups.

6. The wearable radiometer apparatus of claim 1, wherein the garment is a stretchable torso suit, and wherein the antennas are configured to monitor separate locations on the torso.

7. The wearable radiometer apparatus of claim 1, further comprising a data recorder for recording as a function of time the microwave data from the radiometer.

8. A wearable brown adipose tissue activity monitor comprising:
the radiometer apparatus of claim 1;
a separate physical activity monitor;
wherein the radiometer is operatively connected to the activity monitor, and wherein the scanning rate of the radiometer is adjusted based on the level of physical activity determined by the physical activity monitor.

9. A kit comprising:
the wearable radiometer apparatus of claim 1; and
a microwave shielding garment configured to fit thereover.

10. A method of monitoring the activity of brown adipose tissue comprising:
fitting the garment of the wearable radiometer apparatus of claim 1 on a subject with one or more of the antennas of the radio fitted to detect radiation from the brown adipose tissue and one or more other such antennas fitted to detect radiation from tissue that is not brown adipose tissue;
recurrently activating the radiometer to take readings of the temperature associated with the brown adipose tissue; and
outputting from the wearable radiometer a representation of the temperature associated with the brown adipose tissue over time.

11. The method of claim 10, further comprising:
operating a separate physical activity monitor of the subject; and
adjusting the timing of the activating of the radiometer based on the physical activity detected by the separate physical activity monitor.

12. A method of monitoring the metabolic activity of cancer tissue comprising:
fitting the garment of the wearable radiometer apparatus of claim 1 on a subject with one or more of the antennas of the radiometer fitted to detect radiation from the cancer tissue and one or more other such antennas fitted to detect radiation from tissue that is not the cancer tissue;
recurrently activating the radiometer to take readings of the temperature associated with the cancer tissue; and
outputting from the wearable radiometer a representation of the temperature associated with the cancer tissue over time.

13. The method of claim 12, wherein the cancer is breast cancer.

14. The method of claim 12, wherein the cancer is cancer of the esophagus, thyroid, prostate, rectum, ovaries, testes, kidney, skin or muscle.

15. The method of claim 12, wherein the cancer is cancer of the lung, colon, gall bladder, endometria or pancreas.

16. A method of heating a lesion that is a tumor, cancer or other hyperplasia comprising:
fitting the garment of the wearable radiometer apparatus of claim 1 on a subject with a phased array of active antennas of the radiometer apparatus fitted to detect radiation from the lesion and one or more control antennas fitted to detect radiation from tissue that is not the cancer tissue;
recurrently activating the radiometer utilizing different phase offsets with the phased array to take readings of the temperature associated with the lesion and determine phase offsets that obtain a relative high value for that temperature, thereby determining a location for the lesion; and
directing microwave energy to the so determined location of the lesion with a phased array of antennas.

17. A method of monitoring the perfusion of tissue susceptible to poor blood circulation comprising:
fitting the garment of the wearable radiometer apparatus of claim 1 on a subject with one or more of the antennas of the radio fitted to detect radiation from the susceptible tissue and one or more other such antennas fitted to detect radiation from tissue that is not the susceptible tissue;
recurrently activating the radiometer to take readings of the temperature associated with the susceptible tissue; and
outputting from the wearable radiometer a representation of the temperature associated with the susceptible over time.

18. The method of claim 17, wherein the susceptible tissue is heart muscle tissue.

19. The method of claim 17, wherein the susceptible tissue is brain tissue.

20. The method of claim 17, further comprising:
operating a separate physical activity monitor of the subject; and
adjusting the timing of the activating of the radiometer based on the physical activity detected by the separate physical activity monitor.
